(19) United States
(12) Patent Application Publication
(10) Pub. No.: US 2017/0052169 A1
(43) Pub. Date: Feb. 23, 2017

(54) METHOD AND SYSTEM FOR ASSEMBLING A MICROFLUIDIC SENSOR

(71) Applicant: Johnson Electric S.A., Murten (CH)

(72) Inventor: Li Bing ZHANG, Sharon, MA (US)

(21) Appl. No.: 15/344,985

(22) Filed: Nov. 7, 2016

Related U.S. Application Data
(63) Continuation-in-part of application No. 14/083,347, filed on Nov. 18, 2013.

(51) Int. Cl. G01N 33/49; G01N 27/327; B01L 3/00

(52) U.S. Cl. CPC G01N 33/4905; B01L 3/502715; B01L 3/502707; G01N 27/3272; B01L 2300/0887; B01L 2300/0645; B01L 2300/161; B01L 2200/12; G01N 2333/974

(57) ABSTRACT

A microfluidic sensor, such as an electrochemical blood test strip, with more accurate measurement comprises a plurality of channels through which a fluid to be tested flows via a capillary action. One or more electrodes are located under the channels. As the fluid flows over the electrodes in the channels, the impedance between the electrodes may be measured to determine fluid properties. In order to increase the accuracy of the measurements, the electrode deposition may be configured to be less than 10 μm in thickness via a printing process with high process consistency, thereby reducing the disruption of the electrode deposition on the fluid flow.

### CROSS REFERENCE TO RELATED APPLICATION

This non-provisional patent application is a continuation-in-part of U.S. patent application Ser. No. 14/083,347 filed on 18 Nov. 2013, for which priority is claimed under 35 U.S.C. §120, the entire contents of which are fully incorporated herein by reference.

### BACKGROUND

Microfluidic sensors are used in many applications to separate, control, and measure small volumes of liquid, often on the order of nanoliters, such as for blood coagulation tests or other medical purposes. For example, the INRatio handheld meter, manufactured by Alere Corporation of Waltham, Mass., uses disposable sensor strips to measure blood clotting time for patients taking the drug warfarin.

Many microfluidic sensors are able to measure fluid properties using a pair of electrodes. By measuring the impedance over time between the electrodes, coagulation time may be determined. For example, U.S. Pat. No. 7,674,616, titled “Device and Method for Measuring Properties of a Sample,” discloses a coagulation inspection device using automatically collected blood samples.

The ability to obtain accurate measurements is an extremely important concern for microfluidic sensors. Measurement accuracy may be affected by many different factors, such as the deposition of the electrodes, which may affect the measured impedance. Thus, there exists a need for a microfluidic sensor with a thin and smooth electrode deposition to achieve a high accuracy in measurement.

In addition, it is desirable to have multiple measurements on a single sensor. With double-sided circuits, more electrodes can be deposited onto a single substrate, thus more sensor measurements can be taken. Further with printed through-holes (also called “printed vias” in the printed electronics industry), common electrodes can be connected together, so the number of external connection is reduced. With reduced the electrode leads, the compact sensor can be designed.

### SUMMARY

Some embodiments are directed towards a double-sided microfluidic sensor, such as for an electrochemical blood test strip, with more accurate measurement. The sensor comprises a plurality of channels through which a fluid to be tested flows, defined by a cover layer, spacer layer, and a double-sided electrode layer on hydrophilic substrate. One or more electrodes are located under the channels. As the fluid flows over the electrodes in the channels, the impedance between the electrodes may be measured in order to determine fluid properties. In order to increase the accuracy of the measurements, the electrode deposition may be configured to be less than 10 μm in thickness, to reduce the disruption of the electrode deposition on the fluid flow.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered which are illustrated in the accompanying drawings. These drawings depict only exemplary embodiments and are not therefore to be considered limiting of the scope of the claims.

FIG. 1 illustrates a microfluidic sensor with double-side printed electrodes in accordance with some embodiments.

FIG. 2 illustrates double-side electrodes laid on two surfaces of a single substrate in accordance with some embodiments.

FIG. 3 illustrates electrodes on opposite sides of a single substrate connected via a through-hole in the substrate in accordance with some embodiments.

FIG. 4 illustrates a channel in a microfluidic sensor in accordance with some embodiments.

FIGS. 5A-B illustrate a channel with a thick electrode deposition.

FIGS. 6A-B illustrate a channel in a microfluidic sensor in accordance with some embodiments.

### DETAILED DESCRIPTION

Various features are described hereinafter with reference to the figures. It shall be noted that the figures are not necessarily drawn to scale, and that the elements of similar structures or functions are represented by like reference numerals throughout the figures. It shall also be noted that the figures are only intended to facilitate the description of the features for illustration and explanation purposes, unless otherwise specifically recited in one or more specific embodiments or claimed in one or more specific claims. The figures and various embodiments described herein are not intended as an exhaustive illustration or description of various other embodiments or as a limitation on the scope of the claims or the scope of some other embodiments that are apparent to one of ordinary skills in the art in view of the embodiments described in the Application. In addition, an illustrated embodiment need not have all the aspects or advantages shown.

An aspect or an advantage described in conjunction with a particular embodiment is not necessarily limited to that embodiment and may be practiced in any other embodiments, even if not so illustrated, or if not explicitly described. Also, reference throughout this specification to “some embodiments” or “other embodiments” means that a particular feature, structure, material, process, or characteristic described in connection with the embodiments is included in at least one embodiment. Thus, the appearances of the phrase “in some embodiments”, “in one or more embodiments”, or “in other embodiments” in various places throughout this specification are not necessarily referring to the same embodiment or embodiments.

Some embodiments are directed towards a microfluidic sensor, such as for an electrochemical blood test strip, with more accurate measurement. It is understood that while the illustrated embodiments for the sake of explanation refer to measuring blood coagulation, embodiments may be directed to any other type of microfluidic sensor, including but not limited to those used for DNA analysis, glucose testing, etc.

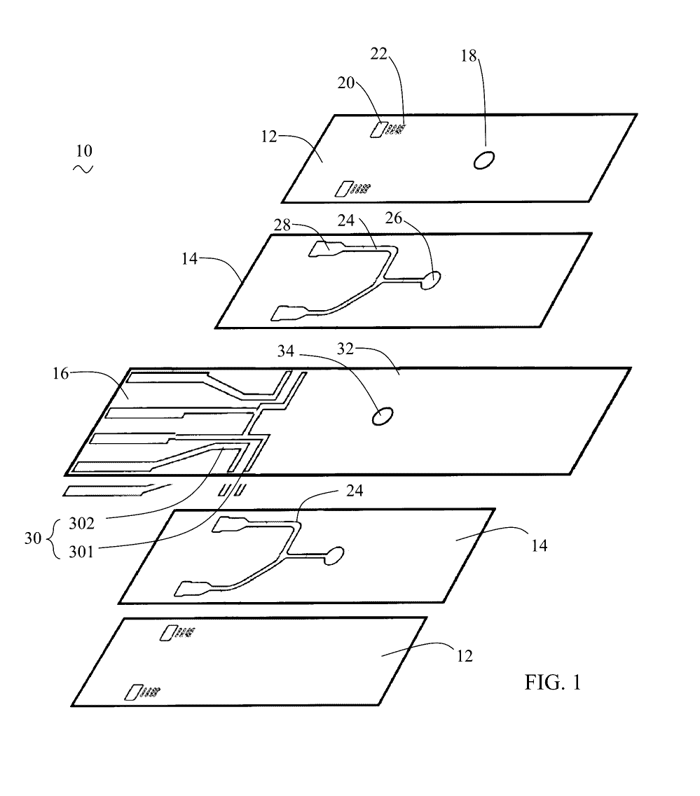

FIG. 1 illustrates a microfluidic sensor 10 in accordance with some embodiments. Sensor 10 may comprise two cover layers 12, two spacer layers 14, and an electrode layer 16. One or more channels 24 may be defined by cut-outs in spacer layer 14, and defined at the top and bottom by cover layer 12 and electrode layer 16, respectively. Electrode layer 16 consists of a hydrophilic substrate 32, electrodes are deposited onto both sides of the substrate. During operation, the fluid to be measured (e.g., blood) travels through channels 24.

Cover layer 12 comprises a protective film or covering (e.g., plastic film) that encloses the top of channels 24. An opening or through hole 18 in cover layer 12 may be used to deposit the fluid into the channels 24. An aligned opening 34 is defined on the hydrophilic substrate 32, so fluid can flow into the channels 24 arranged on both sides of the substrate 32. In addition, cover layer 12 may have a plurality of vent holes 20 formed therein adjacent to the ends of channels 24, to allow the fluid to exit the sensor 10. In some embodiments, measuring a property of the fluid involves subjecting the fluid to a reaction, and cover layer 12 may additionally comprise a plurality of deposit regions 22 where a reagent such as, for example, an enzyme may be deposited. For example, in some embodiments, an enzyme or chemical reagent (e.g., phospholipid) may be used to catalyze a blood coagulation reaction, while other chemical reagents (e.g., sodium citrate) may be used in different embodiments to perform anti-coagulation studies. Deposit regions 22 may be configured to align with reaction zones 28 defined by spacer layer 14, allowing the deposited enzymes and/or other reagents to react with the fluid.

Spacer layer 14 is located between electrode layer 16 and cover layer 12, and contains pre-cut channels 24. The fluid may be deposited into an entry area 26 through entry hole 18 on cover layer 12 and flows into channels 24 through capillary action. Spacer layer 14 also contains a plurality of reaction zones 28 at the end of channels 24 remote from entry area 26. Reaction zones 28 may be configured to interface with deposit regions 22 and vent holes 20 on cover layer 12, allowing enzymes and/or other reagents at deposit regions 22 to react with the fluid, and for the reacted fluid to be able to exit sensor 10 through vent holes 20. In some embodiments, an enzyme is used to catalyze a coagulation reaction, causing the impedance of the fluid (e.g., blood) to change.

In some embodiments, spacer layer 14 defines two channels 24 having a common entry area 26, and each ending at a reaction area 28. It is understood that the number of channels 24 may vary based upon the testing methods and/or measuring mechanisms used.

The thickness of spacer layer 14 defines the height of channels 24. In some embodiments, the thickness of spacer layer 14 is between 50 micrometers (μm) and 100 μm. Spacer layer 14 may be made of a double side adhesive, or a film carrier coated with adhesives on both sides, in order to attach spacer layer 14 between cover layer 12 and electrode layer 16.

Electrode layer 16 comprises an electrode deposition 30 on top of an insulating and hydrophilic substrate 32. The substrate 32 may comprise a flexible dielectric base layer, such as a polyester, polycarbonate, or polyimide plastic film; a hydrophilic coating is applied onto the base film. For aqueous fluids to effectively flow over the substrate, the hydrophilic substrate has a contact angle of <45° with aqueous fluid or water. Preferably, the contact angle is less than 25°. Substrate 32 defines a bottom surface of channels 24, and may be configured to be between 75 μm and 250 μm in thickness. In some embodiments, used for electrochemical analysis, the electrode material for electrode deposition 30 comprises silver/silver chloride, which may be used due to its non-polarizing characteristics. The silver to silver chloride ratio can range from 99:1 to 1:99. For example, the commercially available DuPont 5870 ink is an electrode ink with silver/silver chloride ratio of 80:20. It is understood that other materials types and combinations may also be used for electrode deposition.

Electrode deposition 30 can be deposited on both sides of insulator substrate 32 through an additive process, including but not limited to printing, coating, or spraying. In some embodiments, electrode deposition 30 is deposited using screen printing, wherein a liquid ink is screened through a mesh having a developed circuit pattern, and then deposited onto insulator substrate 32. Following the deposition, the ink may be solidified using a thermal process (e.g., using an IR oven, conveyorized oven, etc.)

In some embodiments, electrode deposition 30 comprises two or more electrodes. For example, in the illustrated embodiment, electrode deposition 30 comprises a transmitting electrode 301, and a receiving electrode 302. The transmitting electrode 301 is configured to be connected to a current source, such as an alternating current (AC) signal generation unit (not shown). An AC signal may be used instead of a DC signal to prevent redox reactions of the electrodes, which may lead to polarization of the electrodes and cause signal drift. During operation, the AC generation unit is configured to provide an alternating current of a predetermined frequency and voltage to transmitting electrode 301. In some embodiments, the AC current is configured to have a voltage of less than 15V and a frequency between 1 kilo-Hertz (kHz) and 100 kHz. Receiving electrode 302 is configured to be connected to a means for receiving a signal (not shown). Transmitting electrode 301 and receiving electrode 302 pass under a channel 24. Thus, during operation, the impedance between transmitting electrode 301 and receiving electrode 302 may be measured and used to calculate the properties of the fluid flowing through channel 24.

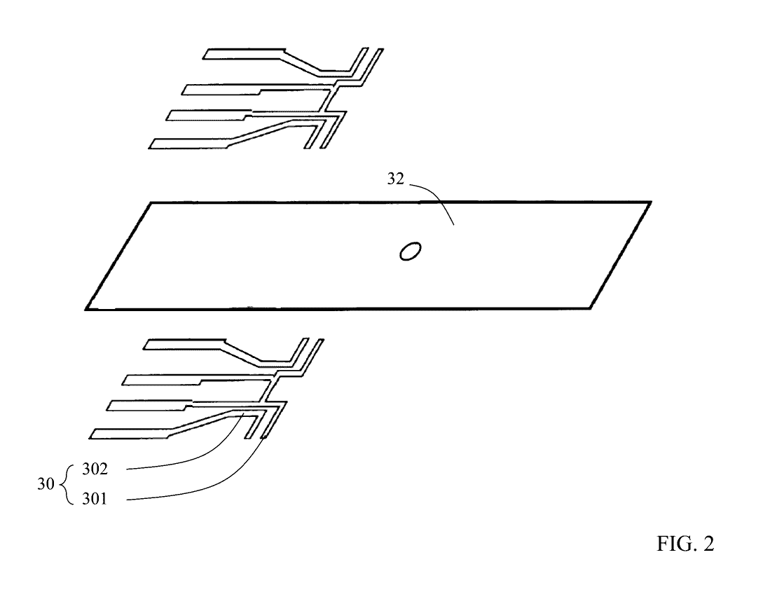

FIG. 2 illustrates double-sided electrodes 30 deposited on substrate 32. The electrode patterns on both sides can be symmetrical or non-symmetrical. With this configuration, a double-side external connector housed in a testing machine is needed in order to make electrical connections to the electrodes on both sides of the substrate. The connector can be spring-loaded type, or inserted type, such as low insertion force (LIF) or zero insertion force (ZIF) connector.

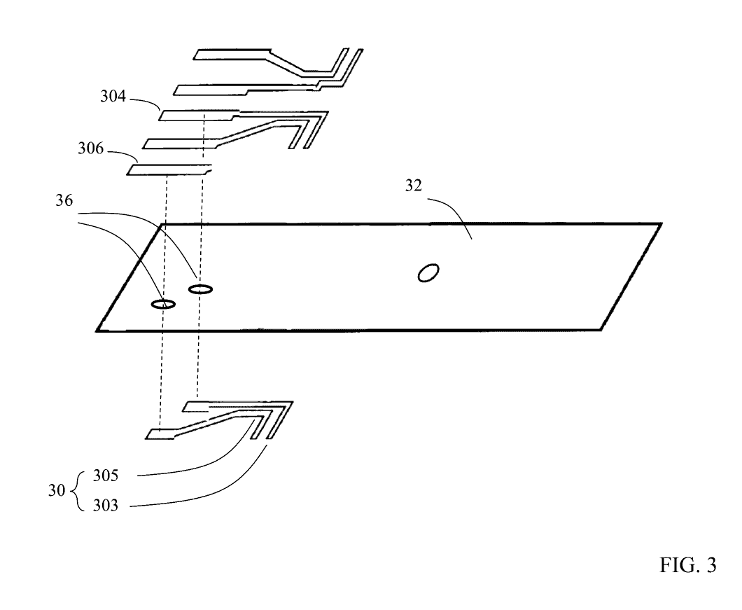

FIG. 3 illustrates double-sided electrodes 30 with through-holes 36 formed on substrate 32. The through-holes 36 can be mechanically or laser drilled on the substrate 32. When electrodes are printed on both sides of the substrate 32, the conductive ink can flow into the through-holes 36 and make electrical connection for traces on both sides of the substrate 32. For certain transmitting and receiving electrode pairs, one of the electrodes can be common electrode, such as ground or reference electrodes. As an example shown in FIG. 3, common electrodes 303 and 304 on the opposite sides of the substrate 32 can be connected using printed through-hole 36. Non-common electrode 305 on the bottom side of the substrate can also be connected through through-hole 36 to a connecting pad 306 on the top side of substrate. With this configuration, only single-sided external connector is needed in order to make electrical connections to the electrodes on top side of the substrate.

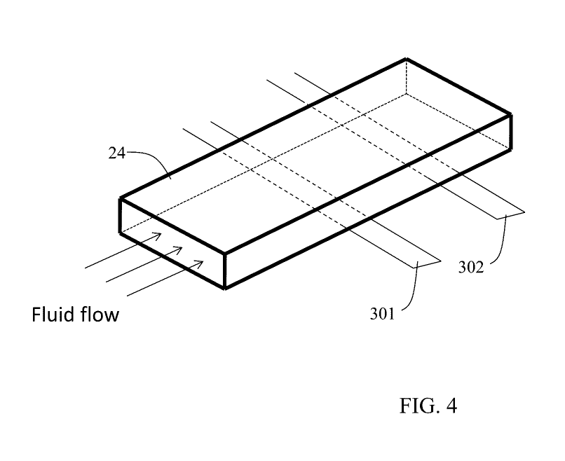

FIG. 4 illustrates a channel 24 that may be used in microfluidic sensor 10 of FIG. 1 in accordance with some embodiments. Channel 24 is defined on the sides by spacer layer 14, on top by cover layer 12, and on the bottom by electrode layer 16. As the fluid flows through channel 24 between entry area 26 and reaction zones 28, it passes over transmitting electrode 301 and receiving electrode 302. Properties of the fluid, such as composition, flow rate, fluid thickness, etc., may affect its impedance between electrodes 301 and 302. Thus, the impedance between electrodes 301 and 302 may indicate properties of the fluid (e.g., flow rate over time, coagulation, etc.) there between.

Fluid volume within reaction zones 28, fluid flow time through channels 24, and distance between electrodes 301 and 302 are critical factors in determining the accuracy of the impedance measurement. Therefore, factors that may impede or disrupt the fluid flow through channels 24 should be minimized. One factor is the thickness of the electrode deposition compared to the height of fluid channel 24. For example, current microfluidic sensors for measuring blood coagulation typically have an electrode deposition thickness of between 10 μm and 20 μm, compared to a total channel height of about 80 μm. When fluid is flowing through the channel, the electrode deposition acts as a barrier and impedes the flow, potentially affecting the rate and volume of the fluid flow, and thus altering the measured impedance. A thick electrode deposition effectively decreases the height of channel 24 when fluid is flowing across the electrodes.

Another factor is the roughness of the surface of the electrode deposition. If the electrode surface exhibits a non-uniform profile, the surface roughness may disrupt the flow of fluid over the electrode surface. This may be especially problematic near the side walls of the channel, where the flow of fluid may be slowed down and potentially cause air trapping.

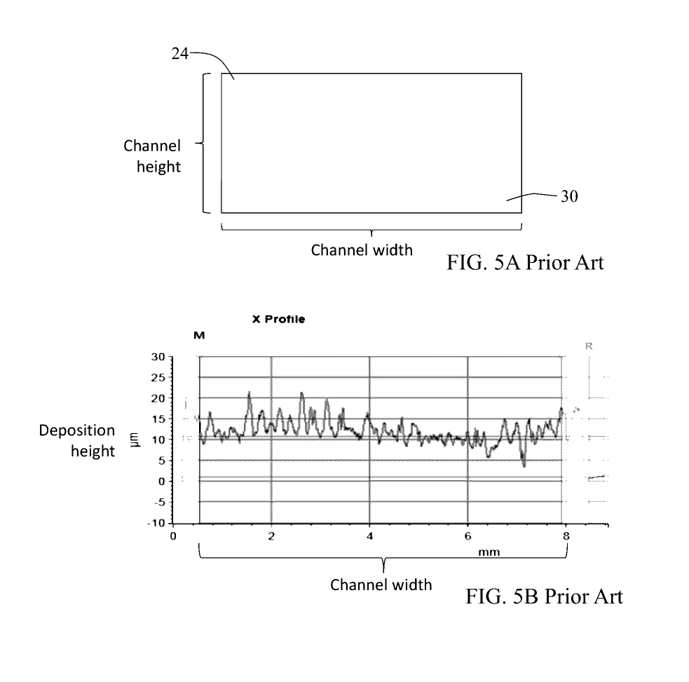

For example, FIG. 5A illustrates a cross section of a channel with a thick electrode deposition using a commercially available ink such as DuPont 5870 ink, along a plane perpendicular to the direction of fluid flow, and FIG. 5B illustrates a surface section scan profile of the electrode deposition along a plane perpendicular to the direction of fluid flow. As can be seen in the figures, the electrode deposition occupies a substantial area of the channel, and has a surface with many peaks and valleys. This acts as a barrier and disrupts the flow of fluid, and thus affecting the measured impedance and calculated fluid properties.

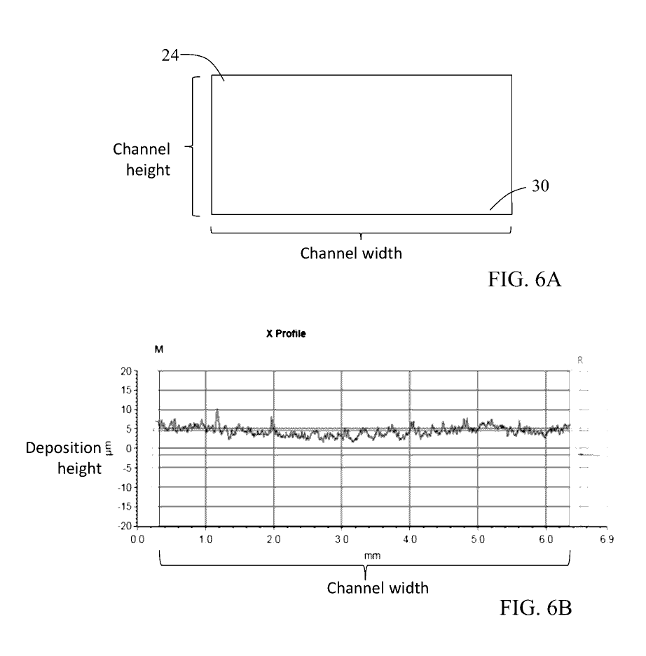

FIG. 6A illustrates a cross section of channel 24, along a plane perpendicular to the direction of fluid flow, wherein the electrode deposition thickness is configured to be less than 10 μm, while FIG. 6B illustrates a surface section scan profile of the electrode deposition 30 along a plane perpendicular to the direction of fluid flow. This may be achieved using certain commercially-available screen-printable conductive inks, such as Parlex ink PF046, available from Parlex USA Inc. of Massachusetts, USA. In accordance with a specific embodiment, the thickness of electrode deposition is configured to be 5 μm or less. As illustrated in FIG. 4A, the thinner electrode deposition 30 allows for more space in the channel 24 for fluid flow, reducing the effects of electrode deposition 30 on the flow. In addition as illustrated in FIG. 4B, the surface of electrode deposition 30 is smoother, with much smaller variation between the peaks and valleys of the deposition, further reducing any disruption of fluid flow by the electrode deposition 30.

By way of example, Table 1 illustrates electrode deposition thickness and its variation of using DuPont 5870 ink and Parlex PF046 ink, wherein the measurements were collected using a micrometer drop gauge, with 40 sample points, and each sample point measures the average thickness of an electrode deposition. Table 1 illustrates the average of the 40 sample points, as well as the minimum and maximum sample points. As can be seen from Table 1, by using screen-printing with the Parlex PF046 ink, a variance of less than 10% for average electrode deposition thickness can be achieved, compared with over 30% variation with the DuPont 5870 ink.

TABLE 1

| Electrode | Average | Maximum | Minimum | |
|---|---|---|---|---|
| Deposition | Thickness | Thickness | Thickness | |
| Ink | (μm) | (μm) | (μm) | Variation |
| Dupont 5870 | 13.9 | 18.5 | 9.5 | +/−32.4% |
| Parlex PF046 | 7.0 | 7.6 | 6.4 | +/−8.6% |

Table 2 illustrates maximum peak to valley variations for a pair of sample electrode depositions, which characterize the roughness of the deposition, using DuPont 5870 ink and Parlex PF046 ink, wherein the measurements were collected using a ContourGT-X8 optical profilometer (Bruker Corporation, Tucson, Ariz., USA). The max X-profile peak to valley variation measures the difference in thickness between the highest peak and lowest valley of an electrode deposition along a plane in the X direction (the width of the channel), while the max Y-profile peak to valley variation measures the distance between the highest peak and lowest valley of the electrode deposition along a plane in the Y direction (parallel to the direction of fluid flow). The data clearly show that the thicker deposition of the DuPont 5870 ink also has a much rougher surface morphology compared to the thinner deposition of PF046 ink.

TABLE 2

| Electrode Deposition | Max Peak to Valley | Max Peak to Valley |
|---|---|---|
| Ink | Variation (X-profile) | Variation (Y-profile) |
| Dupont 5870 | 19 μm | 12 μm |
| Parlex PF046 | 8 μm | 6 μm |

In some embodiments, a prothrombin time (PT) test is used to determine the clotting tendency of blood during warfarin treatment. PT tests may measure tissue factor induced coagulation time of blood or plasma, wherein a prolonged clotting time may suggest the presence of an inhibitor to one or more of the coagulation factors of an extrinsic pathway, or a deficiency in one of the coagulation factors. In some cases, PT time may be prolonged for patients on anticoagulant therapy (e.g., coumarin drug therapy), or for patients with vitamin K deficiency or liver dysfunction. Thus the PT test can provide an assessment of the extrinsic coagulation pathway. During the test, a reagent for measuring PT, such as prothrombin (e.g., recombinant or purified) is deposited at deposition regions 22 on cover layer 12 corresponding to reaction zones 28. Coagulation assays of the PT can be performed independently or in combination with other assays in a reaction zone 28 on microfluidic sensor 10, such as activated clotting time (ACT), activated partial prothrombin time (APTT), thrombin clotting time (TCT), and auto hemolysis test (AHT).

PT test results can be converted to International Normalized Ratio (INR) values according to the formula:

$$INR = {( \frac{{PT}_{test}}{{PT}_{normal}} )}^{ISI}$$

Measured PT results can be plotted to correlate to INR standard, wherein a lower coefficient of variation (CV) of the results may indicate more accurate results. Thus CV values may be used to assess the signal integrity and performance of the sensor. Table 3 illustrates the ratio of CV of INR for a microfluidic sensor using different electrode inks. It can be seen that a thinner, smoother electrode deposition may result in reduced CV and thus more accurate measurements.

TABLE 3

| Electrode Deposition Ink | CV of INR |
|---|---|
| Dupont 5870 | <10% |
| Parlex PF046 | <5% |

In the foregoing specification, various aspects have been described with reference to specific embodiments thereof. It will, however, be evident that various modifications and changes may be made thereto without departing from the broader spirit and scope of various embodiments described herein. For example, the above-described systems or modules are described with reference to particular arrangements of components. Nonetheless, the ordering of or spatial relations among many of the described components may be changed without affecting the scope or operation or effectiveness of various embodiments described herein. In addition, although particular features have been shown and described, it will be understood that they are not intended to limit the scope of the claims or the scope of other embodiments, and it will be clear to those skilled in the art that various changes and modifications may be made without departing from the scope of various embodiments described herein. The specification and drawings are, accordingly, to be regarded in an illustrative or explanatory rather than restrictive sense. The described embodiments are thus intended to cover alternatives, modifications, and equivalents.

1. A double-sided microfluidic sensor, comprising:
A first cover layer;
a second cover layer; and
an electrode layer comprising a substrate having a first surface and a second surface, at least one electrode deposited on the first surface, and at least one electrode deposited on the second surface,
wherein a first channel is formed between the first cover and the first surface of the electrode layer, a second channel is formed between the second cover and the second surface of the electrode layer, and the first channel and the second channel are configured to receive a fluid.

2. The double-sided microfluidic sensor of claim 1, further comprising two spacer layer each of which is located between the electrode layer and a respective one of the first cover layer and the second cover layer, and the first channel and the second channel are defined by cut-outs in the spacer layers.

3. The double-sided microfluidic sensor of claim 1, wherein the substrate of the electrode layer has a thickness less than 10 micrometers (μm).

4. The double-sided microfluidic sensor of claim 1, wherein the at least one electrode deposition on the first surface or the second surface comprises a transmitting electrode and a receiving electrode configured to measure an impedance of the fluid in the first channel or the second channel between the transmitting and receiving electrodes.

5. The double-sided microfluidic sensor of claim 4, wherein the transmitting electrode is configured to receive an alternating current of a pre-determined frequency and voltage.

6. The double-sided microfluidic sensor of claim 4, wherein the impedance is used to calculate coagulation of the fluid.

7. The double-sided microfluidic sensor of claim 1, wherein the at least one electrode deposition is deposited on the substrate of the electrode layer using screen-printing.

8. The double-sided microfluidic sensor of claim 1, wherein a height of the first channel is between 50 μm and 100 μm.

9. The double-sided microfluidic sensor of claim 1, wherein the first cover layer comprises a plurality of through-holes interfacing with the first channel.

10. The double-sided microfluidic sensor of claim 8, wherein the cover layer further comprises a plurality of regions for depositing a reagent to the at least one channel.

11. The double-sided microfluidic sensor of claim 1, wherein the electrodes on both sides of the substrate are electrically connected.

12. The double-sided microfluidic sensor of claim 11, wherein the substrates is formed with through-holes, and the electrodes on both side of the substrate are electrically connected via the through-holes.

13. The double-sided microfluidic sensor of claim 11, wherein the electrodes are formed by printing conductive ink on the substrate, and the conductive ink flow into the through-holes and make electrical connection of the electrodes on both sides of the substrate.

14. The double-sided microfluidic sensor of claim 1, wherein the substrate is a hydrophilic substrate having a contact angle of less than 45° with aqueous fluid or water.

15. The double-sided microfluidic sensor of claim 1, wherein the substrate is a hydrophilic substrate having a contact angle of less than 25° with aqueous fluid or water.

16. A method for assembling and using a double-sided microfluidic sensor, comprising:
depositing a conductive material on a substrate to form at least one electrode on either side of the substrate;
attaching a first surface of a first spacer layer having a first channel defined therein to the substrate with the first channel in the spacer layer passing over the at least one electrode on the substrate layer;
attaching a first cover layer to a second surface of the first spacer layer;
attaching a first surface of a second spacer layer having a second channel defined therein to the substrate with the second channel in the second spacer layer passing over the at least one electrode on the substrate layer;
attaching a second cover layer to a second surface of the second spacer layer.

17. The method of claim 16, wherein depositing a conductive material on a substrate includes depositing an electrically conductive ink.

18. The method of claim 16, wherein the substrate is formed with through-holes, and the electrical conductive ink flow into the through-holes and make electrical connection of the electrodes on both sides of the substrate.

19. The method of claim 16, further comprising forming the substrate by a hydrophilic material to make the substrate having a contact angle of less than 45° with aqueous fluid or water before depositing the conductive material on the substrate.

20. The method of claim 16, further comprising forming the substrate by a hydrophilic material to make the substrate having a contact angle of less than 25° with aqueous fluid or water before depositing the conductive material on the substrate.
